(19) United States
(12) Patent Application Publication
(10) Pub. No.: US 2024/0225696 A1
(43) Pub. Date: Jul. 11, 2024

(54) FOOT CARE APPARATUS

(71) Applicant: Angelo Cavaliere, Cheshire, CT (US)

(72) Inventor: Angelo Cavaliere, Cheshire, CT (US)

(21) Appl. No.: 18/532,058

(22) Filed: Dec. 7, 2023

Related U.S. Application Data
(60) Provisional application No. 63/386,359, filed on Dec. 7, 2022.

(51) Int. Cl. A61B 17/54; A61B 17/32

(52) U.S. Cl. CPC A61B 17/54; A61B 2017/320004

(57) ABSTRACT

The present invention is directed to a foot care apparatus including a base having a first side and a second side, an elongated flap pivotably coupled to the base by a hinge and at least one block extending from either the base or the elongated flap. The elongated flap is pivotable relative to the base so that the foot care apparatus may be positioned in an extended configuration or a folded configuration, and the at least one block is positioned such that an angle between the elongated flap and the base in the folded configuration is between 0 degrees and 90 degrees.

### CROSS-REFERENCE TO RELATED APPLICATIONS

This application claims priority to U.S. Provisional Application No. 63/386,359 filed Dec. 7, 2022, which is hereby incorporated by reference in its entirety.

### BACKGROUND OF THE INVENTION

### 1. Field of the Invention

The present invention is directed to a foot care apparatus, and more particularly to a foot care apparatus configured for cleaning and/or exfoliating of the foot and/or portions thereof.

### 2. Description of Related Art

Foot care includes cleaning, washing, scrubbing, exfoliating, massaging, deodorizing, and otherwise treating the feet. Foot care products may include products and devices for at-home or personal use. However, existing personal foot care products, such as hand-held foot-scrubbing devices for cleaning or exfoliating large surfaces of the feet inconveniently require the use of hands in caring for feet. As a result, a user must either sit down and scrub the feet or stand and try to balance long enough to perform the same. This can be especially problematic in the shower and other surfaces where water on the floor may cause the user to slip and fall. Existing hands-free devices may require the user to exert substantial effort sliding, twisting, and grinding their feet against the product or device. In addition, existing devices may be subject to water or water-related damage, such as warping, mildew formation, etc.

Accordingly, there are a number of disadvantages in conventional foot care devices and products that can be addressed. Therefore, there is a need for a personal foot care device configured for at-home, in-shower use and/or that provides surface stability, hands-free use, and thorough foot care of the foot and portions thereof, such as the heel and/or toes.

### SUMMARY OF THE INVENTION

An exemplary embodiment of the present invention is directed to a foot care apparatus including a base having a first side and a second side, an elongated flap pivotably coupled to the base by a hinge, and at least one block extending from either the base or the elongated flap, the elongated flap is pivotable relative to the base so that the foot care apparatus may be positioned in an extended configuration or a folded configuration, and the at least one block is positioned such that an angle between the elongated flap and the base in the folded configuration is between 0 degrees and 90 degrees.

According to this and other exemplary aspects of the present invention, the foot care apparatus also includes a channel disposed on the elongated flap and positioned on the same side of the foot care apparatus as the second side of the base when the foot care apparatus is in the extended configuration and positioned on the opposite side of the foot care apparatus as the second side of the base when the foot care apparatus is in the folded configuration.

According to this and other exemplary aspects of the present invention, the channel has a substantially arcuate cross-sectional shape.

According to this and other exemplary aspects of the present invention, the channel includes an abrasive surface.

According to this and other exemplary aspects of the present invention, the first side of the base includes a platform having a non-slip surface disposed thereon, and the second side of the base includes an embossed matrix having a non-slip surface.

According to this and other exemplary aspects of the present invention, the elongated flap includes a first side and a second side, and the first side of the elongated flap includes a channel disposed thereon.

According to this and other exemplary aspects of the present invention, the second side of the elongated flap includes the at least one block extending therefrom.

According to this and other exemplary aspects of the present invention, the at least one block is configured to prevent further rotation of the elongated flap relative to the base in the folded configuration of the foot care apparatus.

According to this and other exemplary aspects of the present invention, the elongated flap and the base are comprised of a polypropylene.

According to this and other exemplary aspects of the present invention, when the foot care apparatus is in the folded configuration the second side of the elongated flap and the first side of the base are positioned adjacent to each other and separated by the angle created by the at least one block.

According to this and other exemplary aspects of the present invention, the foot care apparatus is positionable between the extended configuration and the folded configuration by rotating the elongated flap and the base about the hinge relative to one another.

According to this and other exemplary aspects of the present invention, the channel includes a cleaning surface disposed thereon.

### BRIEF DESCRIPTION OF THE DRAWINGS

For a fuller understanding of the nature and objects of the present invention, reference is made to the following detailed description taken in connection with the accompanying drawings in which:

FIG. 1 is a bottom plan view of an exemplary embodiment of a foot care apparatus in an extended configuration according to the present invention;

FIG. 2 is a top plan view of the exemplary embodiment of the foot care apparatus in the extended configuration according to the present invention;

FIG. 3 is a front side view of the exemplary embodiment of the foot care apparatus in the extended configuration according to the present invention;

FIG. 4 is a rear side view of the exemplary embodiment of the foot care apparatus in the extended configuration according to the present invention;

FIG. 5 is a left side view of the exemplary embodiment of the foot care apparatus in the extended configuration according to the present invention;

FIG. 6 is a right side view of the exemplary embodiment of the foot care apparatus in the extended configuration according to the present invention;

FIG. 7 is a top-right perspective view of the exemplary embodiment of the foot care apparatus in the extended configuration according to the present invention;

FIG. 8 is a bottom-right perspective view of the exemplary embodiment of the foot care apparatus in the extended configuration according to the present invention;

FIG. 9 is a bottom-rear perspective view of the exemplary embodiment of the foot care apparatus in the extended configuration according to the present invention;

FIG. 10 is a top-left perspective view of the exemplary embodiment of the foot care apparatus in the extended configuration according to the present invention;

FIG. 11 is a top-front perspective view of the exemplary embodiment of the foot care apparatus in the extended configuration according to the present invention;

FIG. 12 is a bottom-left perspective view of the exemplary embodiment of the foot care apparatus in the extended configuration according to the present invention;

FIG. 13 is a left side view of the exemplary embodiment of the foot care apparatus in a partially extended configuration according to the present invention; and

FIG. 14 is a left side view of the exemplary embodiment of the foot care apparatus in a folded configuration according to the present invention.

### DETAILED DESCRIPTION

The present invention now will be described more fully hereinafter with reference to the accompanying figures, in which exemplary embodiments of the invention are shown. The invention may however, be embodied in many different forms and should not be construed as limited to the embodiments set forth herein. Like reference numerals refer to like elements throughout.

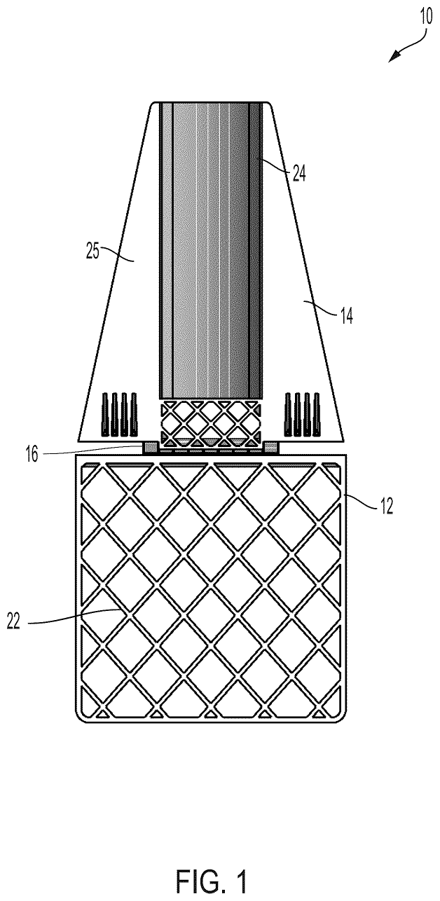

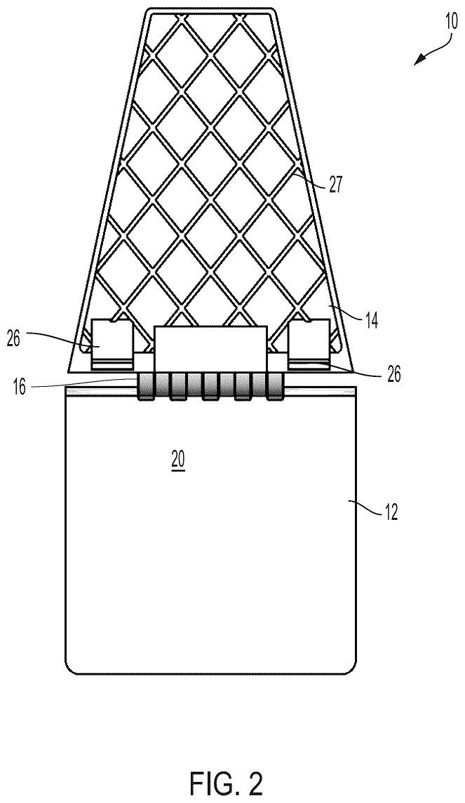

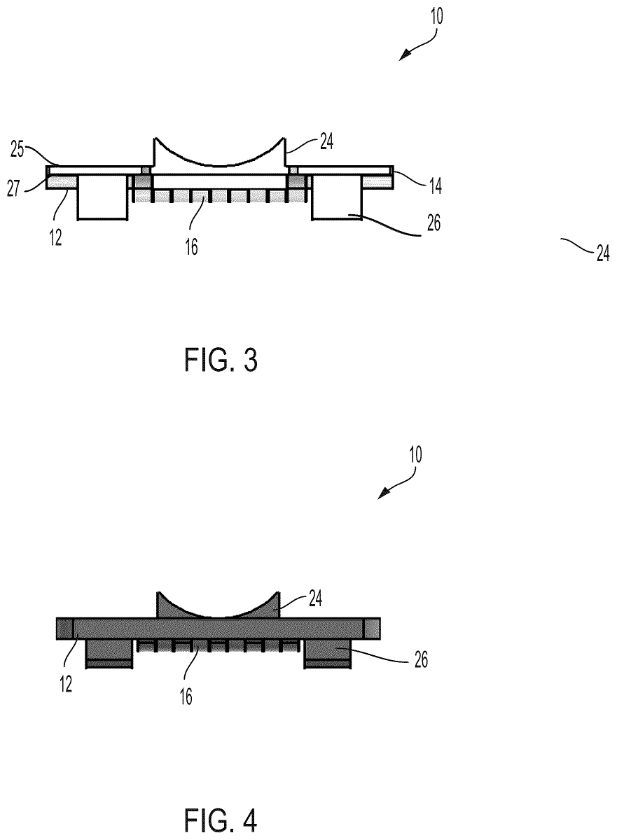

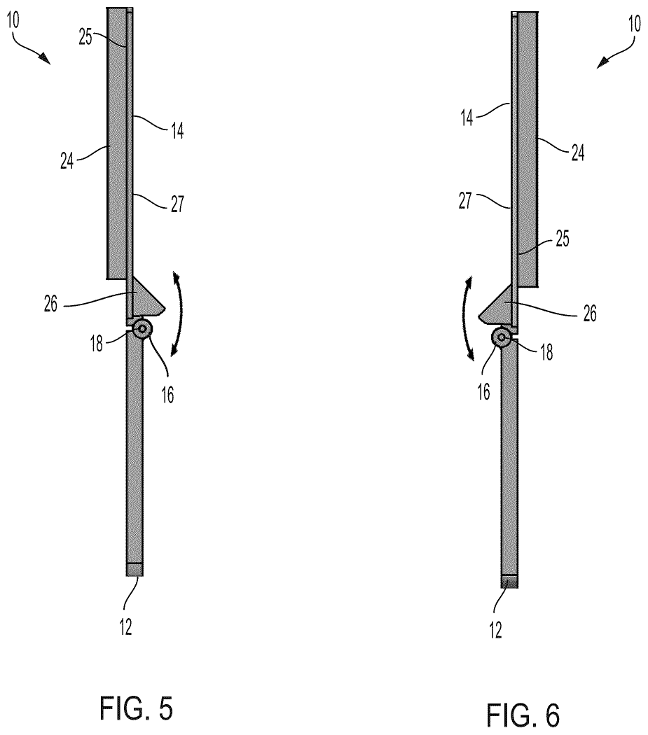

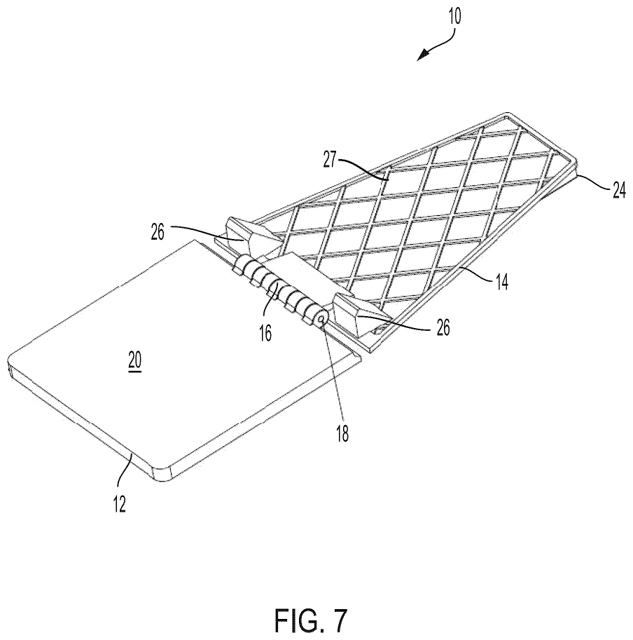

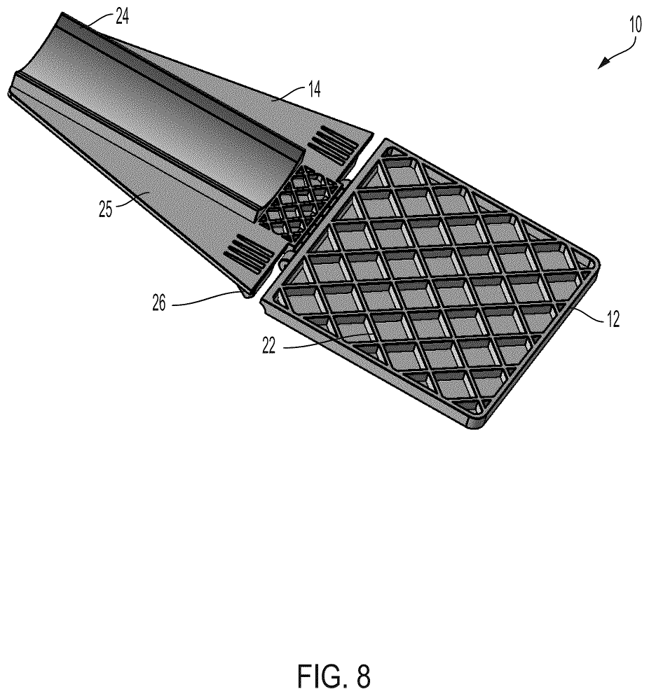

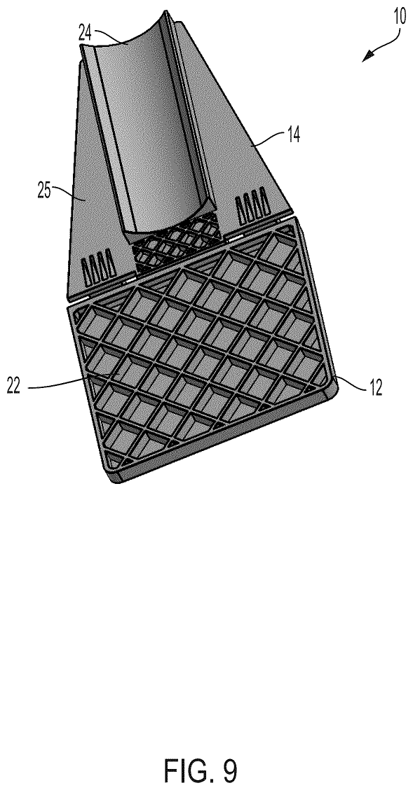

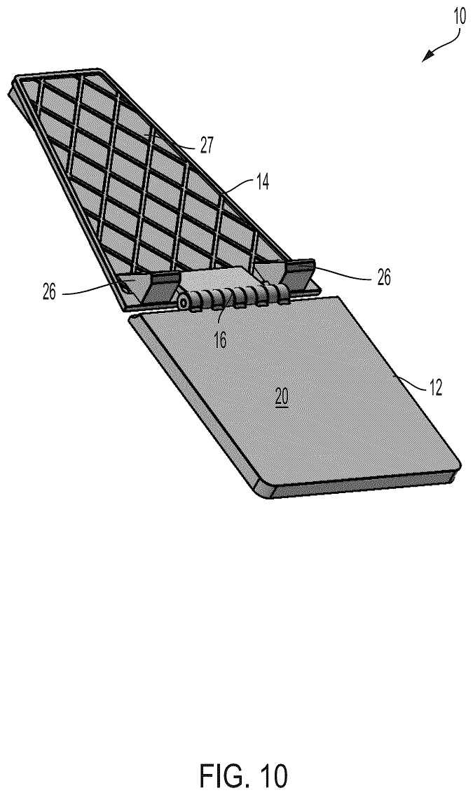

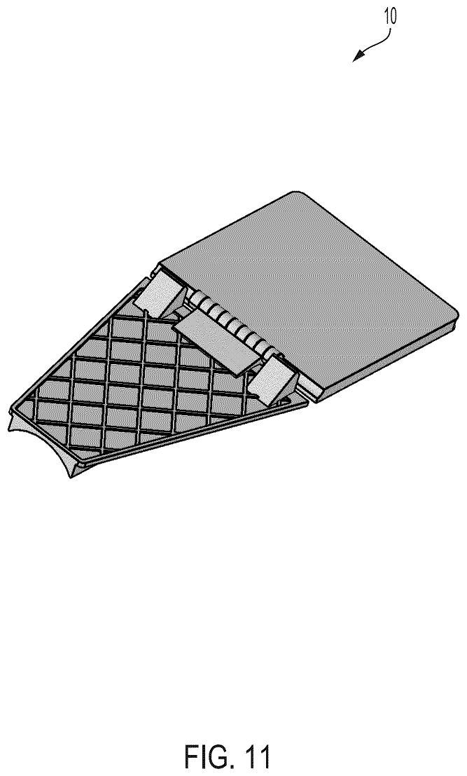

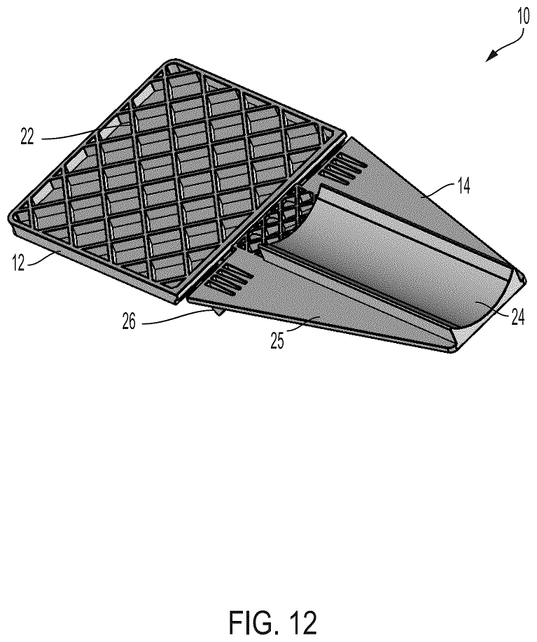

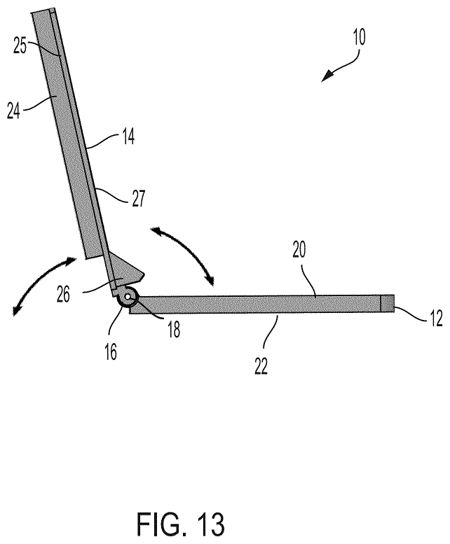

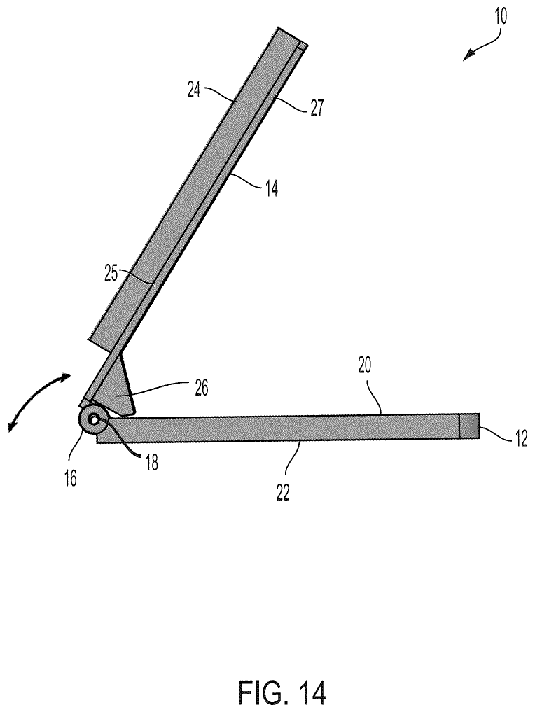

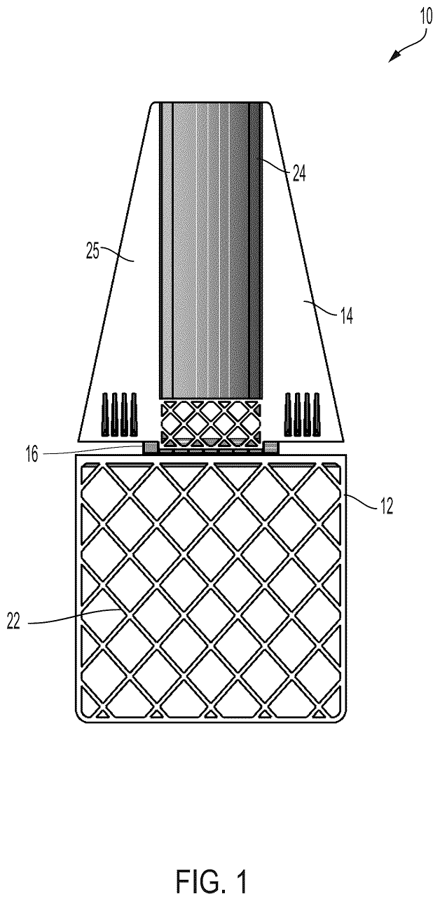

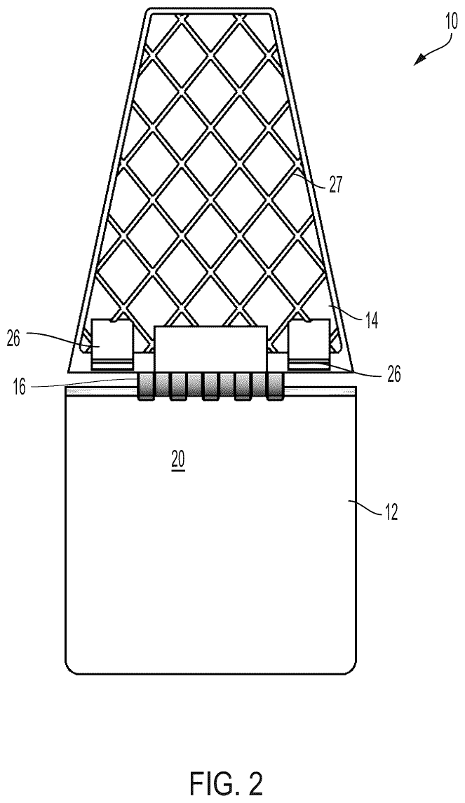

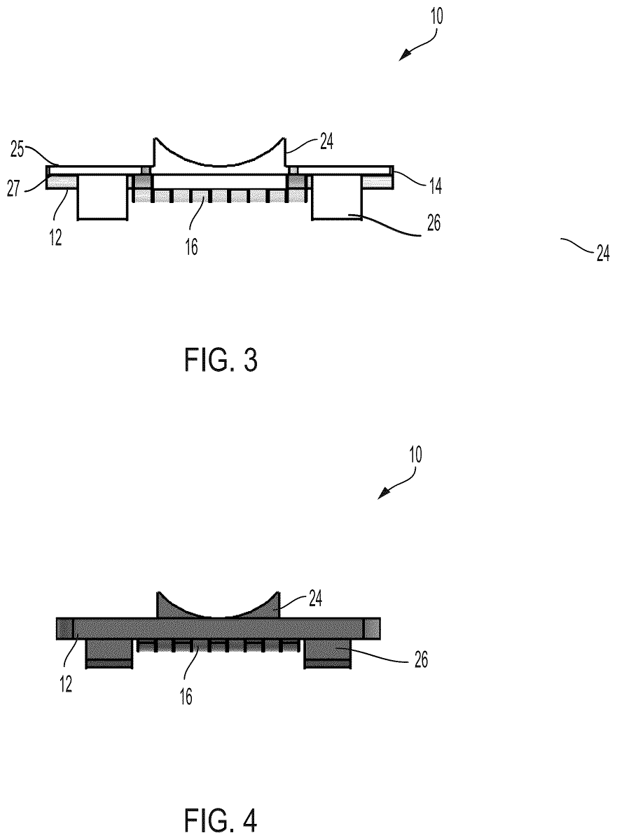

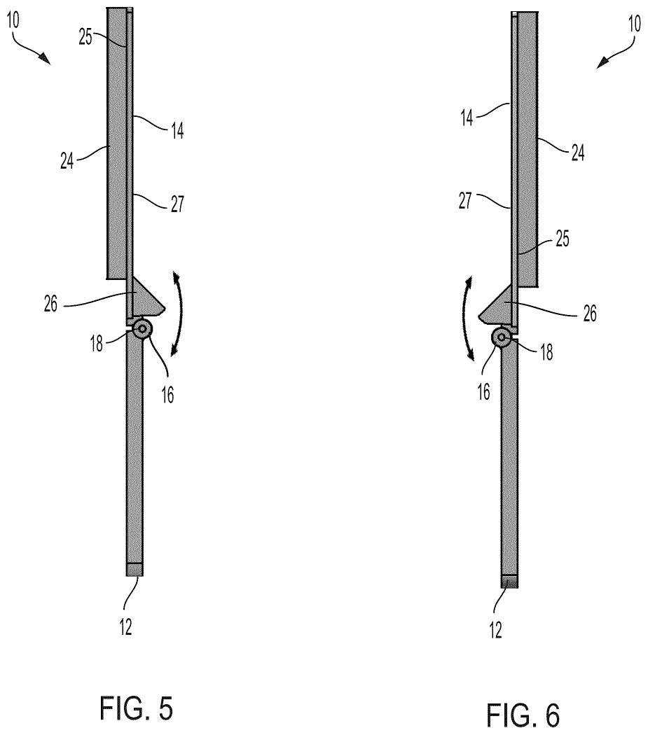

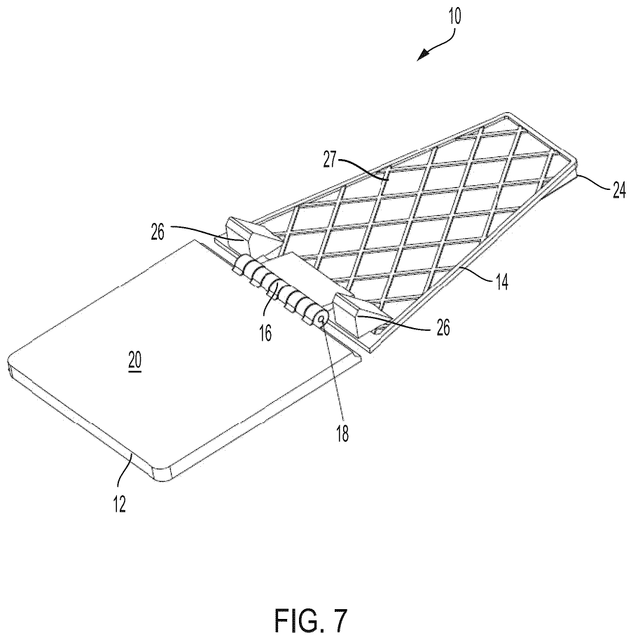

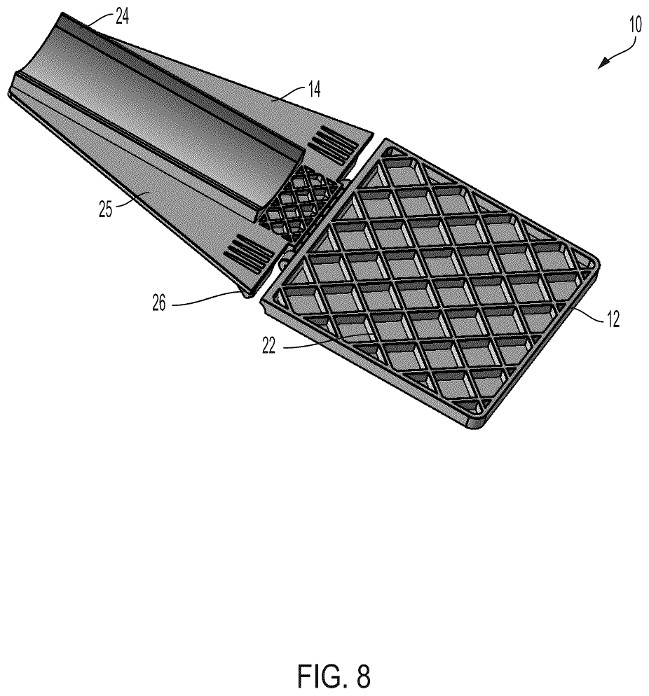

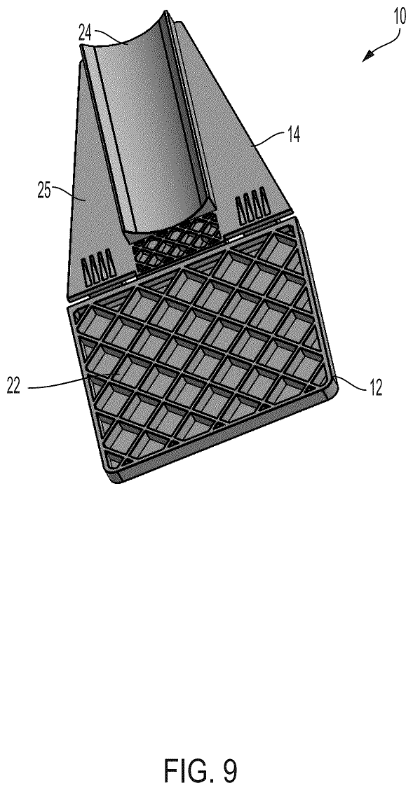

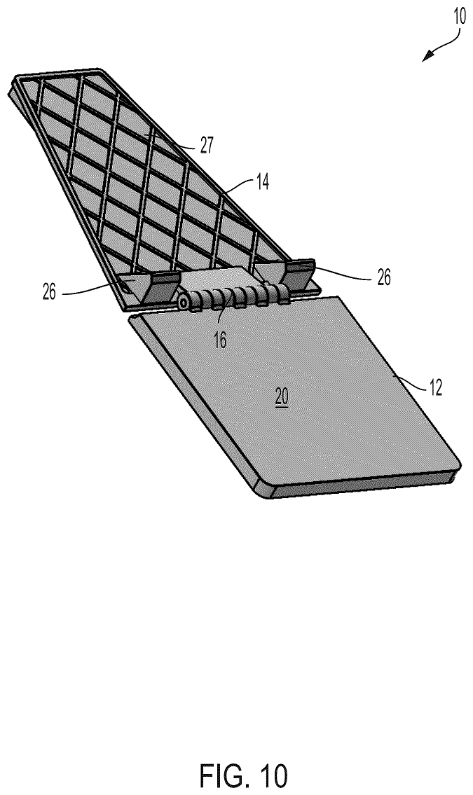

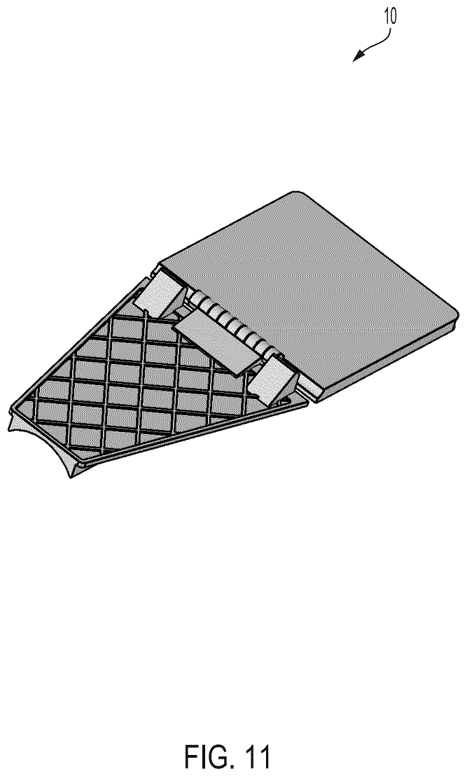

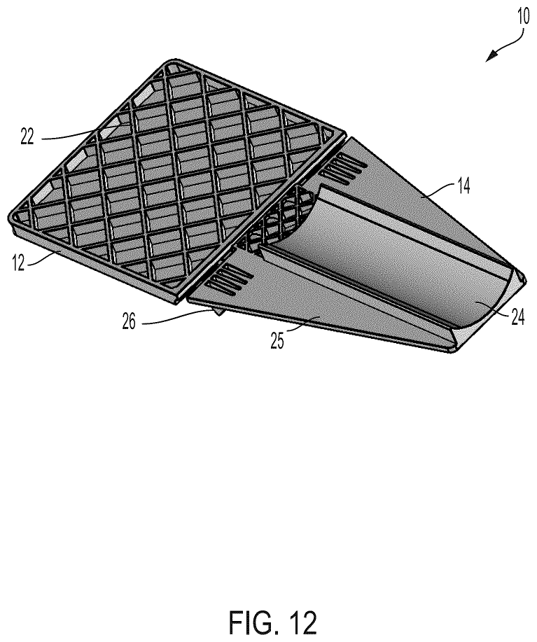

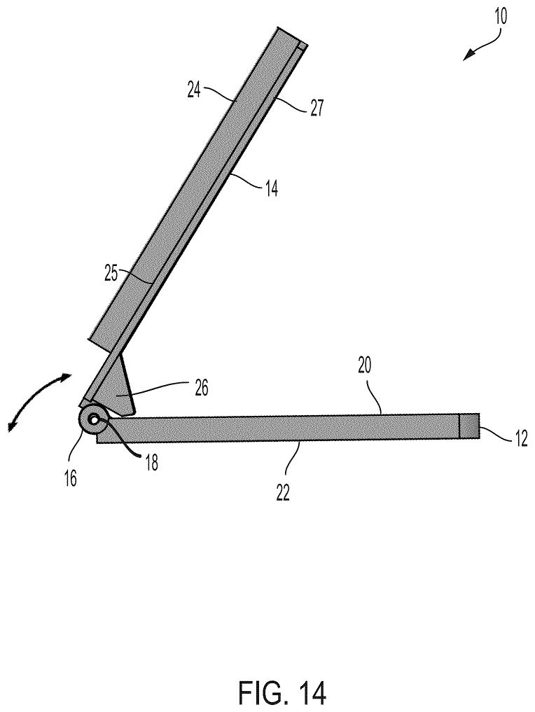

Referring now to FIGS. 1-14, therein illustrated is an exemplary foot care apparatus, generally indicated by reference numeral 10, for use in foot care activities, such as cleaning and/or exfoliating of the feet. The foot care apparatus 10 may include a substantially planar base 12 that may be operatively coupled to an elongated flap 14 by a hinge 16. The hinge 16 is configured to permit rotation of the base 12 and the elongated flap 14 around the longitudinal axis of the hinge 16 so that the foot care apparatus 10 may be positionable between an extended configuration, as shown for example in FIGS. 5 and 6, and a folded configuration, as shown for example in FIG. 14. The hinge 16 may be any suitable construction so as to allow rotation of the base 12 and the elongated flap 14 relative to each other. For example, the hinge 16 may include a pin 18 inserted at least partially through the longitudinal axis of the hinge 16. Respective portions of the hinge 16 may be formed integrally with either the base 12 and the elongated flap 14 and then connected with the pin 18 so that the base 12 and the elongated flap 14 are configured for pivotable rotation relative to each other about the pin 18. Alternatively, the hinge 16 may be formed from a separate component and affixed to the base 12 and the elongated flap 14 by suitable fasteners and/or adhesives. The hinge 16 may also be formed as a living hinge 16 connecting the base 12 and the elongated flap 14. It is understood the hinge 16 may be a single hinge or formed from multiple separate hinges along the connection between the base 12 and the elongated flap 14. It is further understood that the present invention is not limited by any particular construction of the hinge 16, and any suitable construction of the hinge 16 is contemplated by the present invention.

Referring again to FIGS. 1-14, the base 12 may include a platform 20 forming a substantially flat field on a first side of the base 12, and an embossed matrix 22 extending over at least part of a second side of the base 12 opposite the first side of the base 12. The platform 20 may be configured so as to allow a user of the foot care apparatus 10 to place one of the user's feet or a portion thereof, such as one or more toes and/or ball of the foot, on the platform 20 in order to hold the foot care apparatus 10 in place relative to the user. The platform 20 may be coated with a non-slick material (not shown), such as a rubberized and/or textured material, in order to facilitate holding of the foot care apparatus 10 by the user's foot. The embossed matrix 22 is configured for placement on a surface, such as a bathtub or shower floor, opposite of the platform 20. The embossed matrix 22 may similarly be coated with a non-slick material in order to facilitate holding and placement of the foot care apparatus 10 on the surface. It is understood that while the embossed matrix 22 is shown on the second side of the base 12, the present invention is not limited to having embossing on the second side and the second side may be substantially smooth or have a different texture from the exemplary texture shown.

Still referring to FIGS. 1-14, the elongated flap 14 may include a channel 24 positioned on a first side 25 of the elongated flap 14 that is on the same side of the foot care apparatus 10 as the embossed matrix 22 when the foot care apparatus 10 is in the extended position. The channel 24 may have a generally arcuate shape so as to be able to receive one or more portions of the user's foot, such as the heel. The channel 24 may extend from the first side 25 of the elongated flap 14, as in the exemplary embodiment of the present invention shown in the figures, or be recessed into the first side 25 of the elongated flap 14. The channel 24 may include a cleaning surface configured for cleaning and/or exfoliating of the foot and/or portions thereof. In addition or alternatively, the channel 24 may include an abrasive surface, such as a gritted surface from an emery board or a pumice stone, either formed integrally with the arcuate surface of the channel 24 or configured for removable attachment to the arcuate surface of the channel 24. If the channel 24 is composed of an integrally formed abrasive, the entire channel 24 may be removable from the elongated flap 14 in order to affix a new abrasive surface should the previous abrasive surface become less effective and/or fouled. Likewise, if the abrasive surface is configured for removable attachment to the channel 24, the channel 24 may be configured to retain the abrasive surface so that the removable abrasive surface can be replaced when less effective and/or fouled. The elongated flap 14 may also include one or more blocks 26 positioned on a second side 27 of the elongated flap 14 opposite the first side 25 of the elongated flap 14. The one or more blocks 26 are dimensioned and configured to position and retain the elongated flap 14 at an angle relative to the base 12 when the foot care apparatus 10 is in the folded configuration, for example as shown in FIG. 14. The angle of the elongated flap 14 relative to the base 12 may between greater than 0 degrees and less than 90 degrees. As the elongated flap 14 is rotated about the longitudinal axis of the hinge 16 the one or more blocks 26 become closer to and eventually engage with the platform 20 of the base 12 to prohibit further rotation of the elongated flap 14 and base 12 relative to each other. It is understood the present invention is not limited to any particular angle of the folded configuration, and the angle of the folded configuration can be adjusted by the size, position and/or shape of the one or more blocks 26. It is further understood that the one or more blocks 26 may be formed as a continuous block 26 running substantially the length of the elongated flap 14 adjacent to the hinge 16. It is also understood that the one or more block 26 may be positioned on either or both of the elongated flap 14 or the base 12, and that the present invention is not limited to any particular placement or location of the one or more blocks 26 on the foot care apparatus 10.

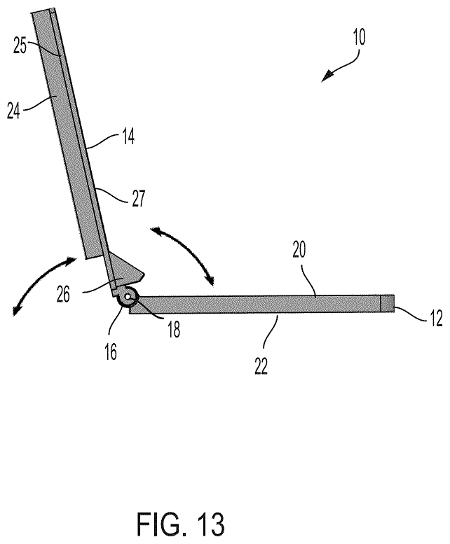

The operation and use of the foot care apparatus 10 will now be discussed with reference to FIGS. 5-6 and 13-14. As discussed above, FIGS. 5 and 6 demonstrate the exemplary embodiment of the foot care apparatus 10 in the extended position in which the base 12 and the elongated flap 14 have been rotated away from each other about the longitudinal axis of the hinge 16. In order to place the foot care apparatus 10 in the folded configuration for use by the user, the elongated flap 14 is rotated towards the base 12, for example as shown in FIG. 13, until the one or more blocks 26 come into contact with the platform 20 of the base 12. In this folded configuration, the elongated flap 14 cannot rotate any further in the direction of the base 12 due to the interference created by the one or more blocks 26 on the platform 20. The elongated flap 14 is now angled relative to the base 12. The base 12 of the foot care apparatus 10 may now be placed on a surface with the embossed side 22 facing down towards the surface so that the channel 24 of the elongated flap 14 is positioned upright. With the channel 24 positioned upright at an angle relative to the surface, the user may now place one foot on the platform 20 in order to hold the foot care apparatus 10 in place and draw the user's other foot along the channel 24 in order to clean and/or exfoliate part of the user's other foot, such as the heel. The user may then switch feet so the foot originally placed on the platform 20 can be cleaned and/or exfoliated by drawing the foot along the channel 24. This action can be performed in a shower if desired.

The base 12 and elongated flap 14 of the foot care apparatus 10 may be constructed of a polypropylene or similar material. The pin 18 may be constructed from a suitable rust-free or rust-resistant metal in order to allow use of the foot care apparatus 10 in wet locations, such as the shower.

It will thus be seen that the objects set forth above, among those made apparent from the preceding description, are efficiently attained and, since certain changes may be made in the above article without departing from the scope of this invention, it is intended that all matter contained in this disclosure or shown in the accompanying drawings, shall be interpreted, as illustrative and not in a limiting sense. It is to be understood that all of the present figures, and the accompanying narrative discussions of corresponding embodiments, do not purport to be completely rigorous treatments of the invention under consideration. It is to be understood that the above-described arrangements are only illustrative of the application of the principles of the present invention. Numerous modifications and alternative arrangements may be devised by those skilled in the art without departing from the scope of the present invention.

What is claimed is:

1. A foot care apparatus, comprising:
a base having a first side and a second side;
an elongated flap pivotably coupled to the base by a hinge;
at least one block extending from either the base or the elongated flap;
wherein the elongated flap is pivotable relative to the base so that the foot care apparatus may be positioned in an extended configuration or a folded configuration; and
wherein the at least one block is positioned such that an angle between the elongated flap and the base in the folded configuration is between 0 degrees and 90 degrees.

2. The foot care apparatus according to claim 1, further comprising a channel disposed on the elongated flap and positioned on the same side of the foot care apparatus as the second side of the base when the foot care apparatus is in the extended configuration and positioned on the opposite side of the foot care apparatus as the second side of the base when the foot care apparatus is in the folded configuration.

3. The foot care apparatus according to claim 2, wherein the channel has a substantially arcuate cross-sectional shape.

4. The foot care apparatus according to claim 2, wherein the channel comprises an abrasive surface.

5. The foot care apparatus according to claim 1, wherein the first side of the base comprises a platform having a non-slip surface disposed thereon, and the second side of the base comprises an embossed matrix having a non-slip surface.

6. The foot care apparatus according to claim 1, wherein the elongated flap comprises a first side and a second side; and
wherein the first side of the elongated flap comprises a channel disposed thereon.

7. The foot care apparatus according to claim 6, wherein the second side of the elongated flap comprises the at least one block extending therefrom.

8. The foot care apparatus according to claim 1, wherein the at least one block is configured to prevent further rotation of the elongated flap relative to the base in the folded configuration of the foot care apparatus.

9. The foot care apparatus according to claim 1, wherein the elongated flap and the base are comprised of a polypropylene.

10. The foot care apparatus according to claim 6, wherein when the foot care apparatus is in the folded configuration the second side of the elongated flap and the first side of the base are positioned adjacent to each other and separated by the angle created by the at least one block.

11. The foot care apparatus according to claim 1, wherein the foot care apparatus is positionable between the extended configuration and the folded configuration by rotating the elongated flap and the base about the hinge relative to one another.

12. The foot care apparatus according to claim 2, wherein the channel comprises a cleaning surface disposed thereon.
